(19) United States
(12) Patent Application Publication
(10) Pub. No.: US 2025/0027159 A1
(43) Pub. Date: Jan. 23, 2025

(54) METHODS AND COMPOSITIONS RELATED TO ALCOHOL USE OR COMPLICATIONS THEREFROM

(71) Applicants: Behavioral Diagnostics, LLC, Coralville, IA (US); University of Iowa Research Foundation, Iowa City, IA (US)

(72) Inventors: Robert Philibert, Iowa City, IA (US); Allan M. Andersen, Iowa City, IA (US)

(21) Appl. No.: 18/776,063

(22) Filed: Jul. 17, 2024

Related U.S. Application Data
(60) Provisional application No. 63/527,302, filed on Jul. 17, 2023.
(60) Provisional application No. 63/594,869, filed on Oct. 31, 2023.
(60) Provisional application No. 63/573,524, filed on Apr. 3, 2024.

(51) Int. Cl. C12Q 1/6883

(52) U.S. Cl. CPC C12Q 1/6883; C12Q 2600/118; C12Q 2600/154

(57) ABSTRACT

This document describes materials and methods for determining whether or not an individual will suffer from severe alcohol withdrawal syndrome or complications therefrom, or seizures related to alcohol withdrawal. This document also describes materials and methods for determining whether or not an individual presenting with myocardial infarction (MI) or acute coronary syndrome is suffering from heavy alcohol consumption (HAC) or alcohol use disorder (AUD).

### CROSS REFERENCE TO RELATED APPLICATIONS

This application claims the benefit of priority under 35 U.S.C. 119 (e) to U.S. Application No. 63/527,302 filed Jul. 17, 2023; U.S. Application No. 63/594,869 filed Oct. 31, 2023; and U.S. Application No. 63/573,524 filed Apr. 3, 2024. These applications are incorporated by reference herein in their entirety.

### FEDERALLY SPONSORED RESEARCH OR DEVELOPMENT

This invention was made with government support under R43AA027197 and R21AA029435 awarded by the National Institutes of Health. The government has certain rights in the invention.

### TECHNICAL FIELD

This disclosure generally relates to methods and compositions for evaluating an individual for heavy alcohol consumption, severe alcohol withdrawal syndrome, or complications associated therewith.

### BACKGROUND

Between 2014 and 2018, over 3.7 million emergency room (ER) visits were made by patients for consideration of alcohol use disorder (AUD) with over 25% of all patients self-report current problem drinking. For each of these patients, clinicians must assess potential for Alcohol Withdrawal Syndrome (AWS), with over 20% of these patients requiring admission. AWS is a syndrome of autonomic dysregulation, neurologic and psychiatric signs and symptoms that can begin a few hours or several days after a reduction or cessation in drinking. Although generally mild, severe AWS can be fatal with hallucinations, delirium and seizures being frequently observed. Unfortunately, there is no generally accepted methods for determining who is at risk for AWS, with only a minority of the admitted patients (between 2-7%) actually developing severe AWS. As a result, clinicians tend to error on the side of caution and admit many patients who could otherwise be managed in less intense setting.

In addition, clinicians have long known that excessive alcohol consumption is a significant risk for coronary heart disease (CHD). Epidemiological studies have shown that the relationship between CHD and alcohol consumption follows a J shaped curve with drinking with increasingly higher risk for CHD as levels of alcohol consumption increase. Unfortunately, a more exacting understanding of this relationship has eluded the field because of the difficulty of establishing reliable diagnoses of CHD in large epidemiologically sound populations and the reliance of often unreliable self-reports of alcohol use as the demarcator of alcohol consumption.

This disclosure provides a number of different solutions to these different problems.

### SUMMARY

Currently, clinicians use their judgement, or indices such as the Prediction of Alcohol Withdrawal Syndrome Scale (PAWSS), to determine whether patients are admitted to hospitals for consideration of alcohol withdrawal syndrome (AWS). Only a fraction of those admitted, however, will experience severe AWS. Previously, we and others have shown that epigenetic methods can quantify alcohol consumption. However, whether these or other alcohol biomarkers, such as carbohydrate deficient transferrin (CDT), could identify those at risk for severe AWS is unknown. To test this, we examined PAWSS scores, DNA methylation, and CDT levels in 125 subjects admitted for consideration of AWS. It was found that PAWSS did not predict severe AWS, nor did it predict seizures. However, methylation sensitive digital PCR (MSdPCR) assessment of cg07375256 and CDT levels did predict severe AWS, with Alc12 methylation also predicting AWS associated seizures (p<7×10⁻⁶). We conclude that epigenetic methods can predict those individuals that are likely to experience severe AWS and/or complications therefrom (e.g., seizures), and that the use of precision epigenetic approaches could eliminate many unnecessary hospitalizations.

In one aspect, methods of determining whether or not an individual will suffer from severe alcohol withdrawal syndrome or complications therefrom is provided. Such methods typically include the steps of determining the methylation status of CpG site cg07375256 in a biological sample from the individual. Generally, the methylation status of the at least one CpG dinucleotide identifies individuals that will suffer from severe alcohol withdrawal syndrome or complications therefrom.

In another aspect, methods of determining whether or not an individual will suffer from seizures related to alcohol withdrawal are provided. Such methods typically include the steps of: determining the methylation status of CpG site cg07375256 in a biological sample from the individual. Generally, the methylation status of the at least one CpG dinucleotide identifies individuals that will suffer from seizures related to alcohol withdrawal.

Generally, an increase in methylation at cg07375256 is indicative of an increased likelihood that the individual will suffer from severe alcohol withdrawal syndrome or complications therefrom or seizures related to alcohol withdrawal. Generally, a decrease in methylation at cg07375256 is indicative of a reduced likelihood that the individual will suffer from severe alcohol withdrawal syndrome or complications therefrom or seizures related to alcohol withdrawal.

In some embodiments, the determining step comprises methylation sensitive digital PCR (MSdPCR). In some embodiments, the determining step includes contacting DNA in the biological sample with bisulfite under alkaline conditions to produce bisulfite-treated DNA; optionally, amplifying the bisulfite-treated DNA to produce amplified bisulfite-treated DNA; contacting the bisulfite-treated DNA with at least one oligonucleotide that is complementary to a sequence comprising the at least one CpG dinucleotide; and detecting the methylation status of the at least one CpG dinucleotide. In some embodiments, the methylation status is determined using bisulfite treated DNA

In some embodiments, the at least one oligonucleotide detects the CpG site in the methylated state. In some embodiments, the at least one oligonucleotide detects the CpG site in the unmethylated state.

In some embodiments, the biological sample is selected from the group consisting of peripheral blood, lymphocytes, urine, saliva, and buccal cells.

In some embodiments, the methods further include assigning a number to the individual based on the Prediction of Alcohol Withdrawal Syndrome Scale (PAWSS).

In some embodiments, the methods further include treating the individual for severe alcohol withdrawal syndrome or complications therefrom or seizures related to alcohol withdrawal. In some embodiments, the methods further include not treating the individual for severe alcohol withdrawal syndrome or complications therefrom or seizures related to alcohol withdrawal.

In still another aspect, articles of manufacture are provided that include at least one nucleic acid oligonucleotide for determining the methylation status of CpG site cg07375256.

In some embodiments, articles of manufacture further include one or more of the following: a second nucleic acid oligonucleotide for determining the methylation status of CpG site cg07375256; reagents for bisulfite-converting nucleic acids; reagents for performing methylation sensitive digital PCR (MSdPCR); phenobarbital; and/or anticonvulsant treatment.

In yet another aspect, computer implemented methods are provided for determining whether or not an individual will suffer from severe alcohol withdrawal syndrome or complications therefrom or seizures related to alcohol withdrawal. Such computer implemented methods typically include obtaining measured data associated with the individual, the measured data comprising the methylation status of CpG site cg07375256; generating a predictive score based on the obtained measured data; and providing a likelihood of severe alcohol withdrawal syndrome or complications therefrom or seizures related to alcohol withdrawal by the individual based on the predictive score.

In some embodiments, such methods further include determining the CpG methylation status for the individual, wherein the methylation status is an indicator of severe alcohol withdrawal syndrome or complications therefrom or seizures related to alcohol withdrawal. In some embodiments, such methods further include outputting a predicted level of severity of the alcohol withdrawal syndrome based on the predictive score.

In yet another aspect, methods of determining whether an individual presenting with myocardial infarctions (MIs) or acute coronary syndrome (ACS) is suffering from heavy alcohol consumption (HAC) or alcohol use disorder (AUD) is provided. Such methods typically include the steps of determining the alcohol T score (ATS) for the individual; and determining ZSCAN25 levels in DNA from the individual (e.g., the ratio of methylation at the cg07375256 loci), wherein the ATS and the ZSCAN25 levels are indicative of whether an individual is suffering from HAC or AUD.

In another aspect, methods of determining the likelihood that an individual presenting with heavy alcohol consumption (HAC) or alcohol use disorder (AUD) will die within about two years are provided. Such methods include the steps of determining the alcohol T score (ATS) (i.e., average of 4 Zscores for cg02583484, cg04987734, cg09935388 and cg04583842) for the individual; and determining the methylation status of CpG site cg07375256 in DNA from the individual (e.g., the ratio of methylation at the cg07375256 loci), wherein the ATS and the methylation status of CpG site cg07375256 are indicative of the likelihood that the individual will die within about two years.

In some embodiments, an increase in the ATS correlates with an increase in the likelihood that the individual will die within about two years. In some embodiments, an increase in methylation at cg07375256 is indicative of an increased likelihood that the individual will die within about two years. In some embodiments, a decrease in methylation at cg07375256 is indicative of a reduced likelihood that the individual will die within about two years.

In some embodiments, the determining step comprises methylation sensitive digital PCR (MSdPCR). In some embodiments, the determining step comprises contacting DNA in the biological sample with bisulfite under alkaline conditions to produce bisulfite-treated DNA; optionally, amplifying the bisulfite-treated DNA to produce amplified bisulfite-treated DNA; contacting the bisulfite-treated DNA with at least one oligonucleotide that is complementary to a sequence comprising the at least one CpG dinucleotide; and detecting the methylation status of the at least one CpG dinucleotide.

In some embodiments, the methylation status is determined using bisulfite treated DNA. In some embodiments, the at least one oligonucleotide detects the CpG site in the methylated state. In some embodiments, the at least one oligonucleotide detects the CpG site in the unmethylated state. In some embodiments, the biological sample is selected from the group consisting of peripheral blood, lymphocytes, urine, saliva, and buccal cells. In some embodiments, the methods further include treating the individual for HAC, AUD, MI and/or ACS.

In still another aspect, articles of manufacture are provided that include at least one nucleic acid oligonucleotide for determining the methylation status of each of CpG sites cg02583484, cg04987734, cg09935388 and cg04583842. In some embodiments, the articles of manufacture further include one or more of the following: a nucleic acid oligonucleotide for determining the methylation status of CpG site cg07375256; reagents for bisulfite-converting nucleic acids; and/or reagents for performing methylation sensitive digital PCR (MSdPCR).

In still another aspect, computer implemented methods are provided for determining the likelihood that an individual will die within about two years. Such methods typically include obtaining measured data associated with the individual, the measured data comprising the methylation status of CpG site cg07375256 and/or the methylation status of cg02583484, cg04987734, cg09935388 and cg04583842; generating a predictive score based on the obtained measured data; and providing the likelihood that the individual will die within about two years based on the predictive score. In some embodiments, the methods further include outputting a predicted level of likelihood based on the predictive score.

In one aspect, methods of determining whether or not an individual will suffer from severe alcohol withdrawal syndrome or complications therefrom are provided. Such methods typically include the steps of determining the methylation status of CpG site cg07375256 in a biological sample from the individual, where the methylation status of the at least one CpG dinucleotide identifies individuals that will suffer from severe alcohol withdrawal syndrome or complications therefrom. In some embodiments, the complications from severe alcohol withdrawal syndrome comprise seizures.

In some embodiments, an increase in methylation at cg07375256 is indicative of an increased likelihood that the individual will suffer from severe alcohol withdrawal syndrome or complications therefrom, while in some embodiments, a decrease in methylation at cg07375256 is indicative of a reduced likelihood that the individual will suffer from severe alcohol withdrawal syndrome or complications therefrom.

In some embodiments, the determining step comprises methylation sensitive digital PCR (MSdPCR). In some embodiments, the determining step comprises contacting DNA in the biological sample with bisulfite under alkaline conditions to produce bisulfite-treated DNA; optionally, amplifying the bisulfite-treated DNA to produce amplified bisulfite-treated DNA; contacting the bisulfite-treated DNA with at least one oligonucleotide that is complementary to a sequence comprising the at least one CpG dinucleotide; and detecting the methylation status of the at least one CpG dinucleotide. In some embodiments, the methylation status is determined using bisulfite treated DNA.

In some embodiments, the at least one oligonucleotide detects the CpG site in the methylated state. In some embodiments, the at least one oligonucleotide detects the CpG site in the unmethylated state.

In some embodiments, the biological sample is selected from the group consisting of peripheral blood, lymphocytes, urine, saliva, and buccal cells.

In some embodiments, the methods further include assigning a number to the individual based on the Prediction of Alcohol Withdrawal Syndrome Scale (PAWSS).

In some embodiments, the methods further include treating the individual for severe alcohol withdrawal syndrome or complications therefrom. In some embodiments, the methods further include not treating the individual for severe alcohol withdrawal syndrome or complications therefrom.

In another aspect, methods of determining whether an individual presenting with myocardial infarction (MI) or acute coronary syndrome (ACS) is suffering from heavy alcohol consumption (HAC) or alcohol use disorder (AUD) are provided. Such methods typically include the steps of determining the alcohol T score (ATS) (i.e., average of 4 Zscores for cg02583484, cg04987734, cg09935388 and cg04583842) for the individual; and determining ZSCAN25 levels in DNA from the individual (i.e., the ratio of methylation at the cg07375256 loci), where the ATS and the ZSCAN25 levels are indicative of whether an individual presenting with myocardial infarction (MI) or acute coronary syndrome (ACS) is suffering from HAC or AUD.

In some embodiments, the determining step comprises methylation sensitive digital PCR (MSdPCR). In some embodiments, the determining step comprises contacting DNA in the biological sample with bisulfite under alkaline conditions to produce bisulfite-treated DNA; optionally, amplifying the bisulfite-treated DNA to produce amplified bisulfite-treated DNA; contacting the bisulfite-treated DNA with at least one oligonucleotide that is complementary to a sequence comprising the at least one CpG dinucleotide; and detecting the methylation status of the at least one CpG dinucleotide. In some embodiments, the methylation status is determined using bisulfite treated DNA.

In some embodiments, the at least one oligonucleotide detects the CpG site in the methylated state. In some embodiments, the at least one oligonucleotide detects the CpG site in the unmethylated state.

In some embodiments, the biological sample is selected from the group consisting of peripheral blood, lymphocytes, urine, saliva, and buccal cells. In some embodiments, wherein the methods further include treating the individual for HAC, AUD, MI and/or ACS.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a plot showing the correlation between the Illumina probe assessment and the MSdPCR assessments at the Alc12 locus (n=90) using the Dcg07375256 assay. Overall, the r=0.92 with the dynamic range of the ddPCR is considerably greater than that of the methylation array.

FIG. 2 is a plot showing the correlation of study variables to each other. *=p<0.05, ** p<0.01, *** p<0.001. Age is expressed in years. Sex is binary variable whether or not the subject is male. Drinks scale is an ordinal variable with higher score indicating greater intake. PAWSS is Prediction of Alcohol Withdrawal Syndrome Scale. BAC>200 indicates if a breathalyzer value greater than 200 mg/dl was noted at admission. CDT is carbohydrate transferrin levels. cg05575921 is DNA methylation at that smoking related locus. ATS is Alcohol T Score. ZSCAN25 is DNA methylation per the Dcg07375256 described herein. Diazepam equivalents is the sum total equivalent of all benzodiazepines administered during hospitalization for AWS. Phenobarbital is a binomial variable indicating whether or not phenobarbital was given during hospitalization for AWS. Seizure and hallucinations are binomial variable for the presence of those symptoms.

FIG. 3A-3C are plots showing the relationship of predictor (PAWSS (3C), ATS (3B) and ZSCAN (Dcg07375256) (3A)) values to treatment group. Because control subjects were not administered the PAWSS, their values are not considered in that section of the analyses.

FIG. 4A-4C are plots showing the relationship between predictor levels and presence (1) or absence (0) of seizures. N=9 for the seizure group, 116 for the no seizure group. For Panel 4A, the value for the comparison only between cases with and without seizures for ZSCAN25 (Dcg07375256)methylation is 6.3×10⁻⁶. For Panel 4B, the value for the contrast only between cases with and without seizures is p<0.01.

FIG. 5A-5B are graphs showing the distribution of ZSCAN25 and alcohol T score (ATS) in acute coronary syndrome (ACS) subjects. Normal levels of ZSCAN25 in abstinent individuals are 25.3±4.5%, while <3% of self-reported abstinent individuals have an ATS>3.5. Arrows indicate the lower bound of predictive ranges for severe alcohol withdrawal syndrome (AWS) (FIG. 3A) and heavy alcohol consumption (HAC) (FIG. 3B). Nearly 20% have ZSCAN25 levels predictive of severe AWS with 5 of those subjects in the range associated with seizures. Note that the ACS subject with ZSCAN25 of 84% has an ATS of nearly 19, which is the highest that we have ever observed.

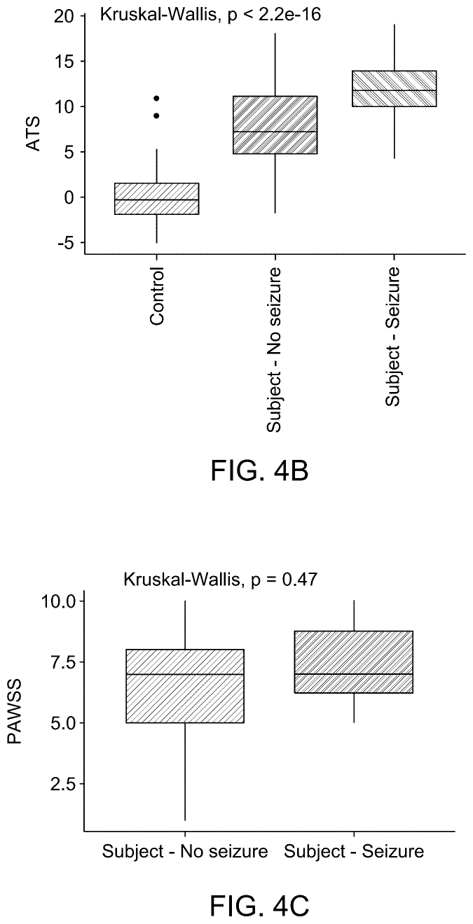

FIG. 6 is a graph showing the distribution of ZSCAN25 methylation in the cohort (n=165).

FIG. 7 is a graph showing the distribution of the Alcohol T Score (ATS) in the cohort (n=170). Values greater than the red line (3.5) are highly predictive of current heavy alcohol consumption (HAC).

### DETAILED DESCRIPTION

Recent progress in DNA methylation biomarkers may have made a more granular, quantitative understanding of the relationship of alcohol consumption to the development of CHD achievable. First, a metric known as the Alcohol T Score (ATS), which is formed using the data from four methylation sensitive digital PCR (MSdPCR) assays, is capable of sensitively and specifically detected heavy alcohol consumption (HAC), which, for these purposes, is defined as drinking six or more drinks per day for eight consecutive weeks. It has been demonstrated that the ATS outperforms the CDT in predicting HAC, accelerated aging and alcohol related immune cell changes. This metric for long-term consumption of alcohol also can be complemented by a marker for short term alcohol consumption that is capable of predicting the occurrence of severe alcohol withdrawal syndrome (AWS) and AWS related seizures.

The second area of progress has been in DNA methylation biomarkers for CHD itself. Specifically, a sensitive and specific Artificial Intelligence (AI) algorithm has been developed that interprets the genetically contextual signal from six MSdPCR assays and validated it in three independent cohorts. Critically, each of these six MSdPCR assays map to unique potentially modifiable pathways known to be involved in the development of CHD.

These advances are timely because CHD is the leading cause of death in the United States and up to 80% of all CHD events are thought to be potentially preventable. However, in order to achieve effective prevention of CHD events, the proximal and distal drivers of CHD and CHD events must be identified. With this aim in mind, structural equation modeling was used to examine the relationship of postulated psychosocial drivers of CHD to changes in DNA methylation at these six CHD related CpG sites using clinical and biological data from the Family and Community Health Studies (FACHS). Interestingly, it was found that exposure to discrimination and insecurity of African Americans as youths strongly predicted the development of excessive alcohol consumption, which, in turn, strongly predicted CHD related changes at these six loci. Strikingly, only objective measures of alcohol use, obtained from methylation and carbohydrate deficient transferrin assessments, but not self-report of alcohol use, predicted CHD development. Taken in conjunction with epidemiological findings, these data suggest that cryptic alcohol use may be a major driver of the development of CHD.

Research settings are not the only places that self-report of alcohol use may be unreliable. In a study of AWS, it was found that, while objective assessments of alcohol consumption obtained via blood alcohol concentrations, methylation and carbohydrate deficient transferrin assessments at admission were highly correlated with one another, none of them were correlated with self-report of alcohol consumption. Given the findings with the FACHS, with unreliable self-report of alcohol use in clinical settings by others, and with our own clinical experiences, this led us to question whether self-reports of alcohol use in those admitted for acute coronary syndrome were reliable.

According to the American Society of Addiction Medicine (ASAM), the use of clinician administered scales such as The Prediction of Alcohol Withdrawal Severity Scale (PAWSS) or the Lübeck Alcohol withdrawal Risk Scale (LARS) can be used for assessing risk of severe AWS. However, in their review of 530 studies of AWS, Wood et al. (2018, JAMA, 320 (8): 825-33) noted a potential for bias and a lack of independent validation of these scales. Hence, there is a strong need for either independent validation of the clinical utility of these tools, or, if deemed not useful, the derivation of new measures capable of helping clinicians decide whether to admit a prospective patient for more intensive monitoring and/or treatment.

Advances in our understanding of the effect of alcohol on the methylome may help. Specifically, in prior work, we have shown that an index composed of 4 methylation sensitive digital PCR assays (MSdPCR), referred to as the Alcohol T Scale, is strongly correlated with other objective biomarkers of heavy alcohol consumption (HAC) and can be used to accurately classify those admitted for residential treatment of alcohol use disorder (AUD). However, whereas this tool is useful for predicting both chronic HAC and the biological consequences of HAC, the dynamic response time of the four loci is slow, with a half-time of reversion of the most responsive locus, cg04987734, being on the order of three months. If the capacity to experience alcohol withdrawal can develop in less time, however, these loci may be too insensitive to be clinically useful for predicting AWS.

There are two key barriers to the development of a better tool for predicting severe AWS. The first barrier is that the minimum amount and timing of alcohol consumption necessary to establish vulnerability to AWS is known. Using an experimental design that would not pass current ethical standards, Isbell and colleagues enrolled ten prison convicts in a study that had them ingest between 286 and 489 of 95% ethanol, based on body weight, for a variable period of at least 7 and up to 87 days. Those who drank less than 34 days experienced clear signs of what we now term AWS, but no seizures or delirium tremens. However, of the six subjects who drank between 48 and 87 days, two had seizures and three had frank delirium. Similarly, in the early 1960's, Mendelson and Ladou had ten chronic alcoholics, without a history of seizure and who had been abstinent for between 10 and 37 days, ingest an average of 30 ounces of 86 proof alcohol for 24 consecutive days, then monitored them for signs of alcohol withdrawal. Although numerous symptoms of AWS were noted to be present, no seizure nor frank delirium were observed. Critically, both sets of investigators took pains to ensure their subjects had adequate nutrition and it is important to note that the use of other substances that might affect likelihood of severe AWS were not permitted. Therefore, although these studies have small, biased samples, they do indicate that a sustained period of heavy drinking for at least a couple of weeks is necessary to induce vulnerability to AWS.

The second barrier to developing better tool for predicting AWS is that, even if we could establish the minimum dose and timing of alcohol necessary to become vulnerable to AWS, the ability of patients to accurately report their prior alcohol intake in clinical settings is less than optimal. This problem is particularly exacerbated in the ER setting when involuntary hospitalization for AWS is being considered. Therefore, any clinical tool solely based on self-report inherently will have a high failure rate.

Conceivably, a biomarker that reflects the amount and/or intensity of alcohol use could be used to predict AWS. Currently, both phosphatidyl ethanolamine (PEth) and carbohydrate deficient transferrin (CDT) assays are used to assess recent alcohol use. Recently, in 34 patients admitted for treatment of AWS, Novak et al. (2023, Alcohol Alcohol, 58 (2): 198-202) found that PEth levels were only modestly associated with severity of AWS and did not predict severe AWS. Furthermore, Helander et al. (2019, Alcohol Alcohol, 54 (3): 251-7) note considerable variation in PEth catabolismthat may make it less suitable as a biomarker for predicting AWS. Similarly, examinations of CDT have shown only modest power to predict severe AWS.

One possible problem for using these two biomarkers to predict AWS may be the relative short half-life of the two biomarkers. PEth has an elimination half-life of 4 to 10 days, while CDT has a half-life of between 7 and 10 days. If alcohol consumption and/or the window for assessing alcohol consumption need to be longer, these assays may not be suitable.

The methods and compositions described herein are based on the identification of methylation markers with reversion half-lives considerably less than those contained in the ATS. The experiments described herein examine whether one or more of these markers are capable of predicting AWS.

We provide strong evidence herein that methylation at cg07375256, a CpG site in the SCAN25 gene (at Chromosome 7, base pair 99,222,196 in CRCh37/hg19 build; also referred to herein as Alc12), is associated with seizures and the decision to administer phenobarbital to patients who have been admitted for observation and/or treatment of AWS. It is noted that the results herein are based on a single site study of largely White subjects from the Midwest United States and will be confirmed with a variety of diverse populations.

A hallmark of AWS is autonomic instability. Therefore, the finding that cg07375256 maps to the transcription start site region of ZSCAN25, a gene previously associated with hypertension, maps well to our clinical understanding of AWS. Still, it is important to realize that associations are not causations, and that even in our examination of entry and exit methylation in 45 pairs of DNA samples, methylation at a number of other loci also significantly reverted during the three-week period of hospitalization-induced abstinence and that the methylation changes observed were in WBCs, not the neuronal or vascular tissue mediating the autonomic instability. Therefore, a truly thorough understanding of the relationship between changes in DNA methylation and the clinical signs and symptoms of AWS will require additional integrated animal model and human studies. Still, the data presented herein demonstrating that cg07375256 methylation can predict severe AWS is strong.

The relatively short time to achieve 50% reversion of methylation at cg07375256 may suggest the reason why other biomarkers have failed to predict serious AWS. The CDT and Peth have relatively short half-lives, on the order of 7-10 days, and 4-10 days, respectively. Although the earlier work of Isbell and Mendelson groups is somewhat limited by current standards, they do suggest that long periods of sustained heavy drinking, on the order of 15 drinks per day for more than 3 weeks, is necessary to become vulnerable to serious AWS. If so, the short window of time recognized by these biochemical methods may be insufficient to capture the critical period of usage. At the other extreme, the markers in the ATS, which was calibrated to predict chronic HAC, all have reversion half-lives of 3 months or more, but only modestly predict seizures and do not predict the likelihood of the patient receiving phenobarbital, which suggests that their window of detection is too long. However, methylation at cg07375256, which has a tentative half-life of reversion of approximately 3 weeks, may serve as a more sensitive barometer for the critical period of sustained alcohol consumption necessary for induction of AWS vulnerability.

At first glance, the finding that PAWSS values were associated with total benzodiazepine dose and BAC levels is intriguing. However, the PAWSS specifically asks whether patients have used downers, including benzodiazepines, been treated for AWS and have a BAC level of greater than 100 mg/dl. As such, the positive association of the PAWSS with these two variables may just reflect internal self-validity of the scale, or alternatively, the tendency of patients who have received benzodiazepines in the past to manifest behaviors that they are administered them in the future. Indeed, when the two above PAWSS questions are removed from the index, there is no significant relationship between PAWSS scores and benzodiazepine usage.

A striking finding is the lack of association of seizures, the use of phenobarbital and any of four biological markers of alcohol consumption (i.e., BAC, ATS, CDT, and cg07375256) with subject reports of alcohol consumption. Although other interpretations are possible, the most parsimonious explanation is that the subject reports of their alcohol consumption are not reliable. Indeed, in our clinical experience and that of others, we have found that patients often misrepresent their alcohol use history. Although others may disagree, in our research experience, we have routinely found stronger relationships between biomarkers of alcohol use to one another than to that of self-report. When considering the circumstances in which most of the subjects present, this is certainly understandable. Given the fact that many of the subjects did not willingly present for treatment and may fear the information being used against them, they may be reluctant to fully disclose their past usage of alcohol. Or even more understandably, the subjects may not remember. For example, even under the best of circumstances, dietary reports by sober patients have high rates of error. Given the level of intoxication that many of these patients report, they simply may not recall how much they have been drinking or how long they have been drinking. In any case, it is clear that the biological variables in this study tend to be positively associated with one another, while self-report of alcohol use only correlates with, unsurprisingly, scales that are based in part on self-report.

An interesting question is why has this marked association has not been observed previously? In some respects, perhaps it already has been noted. In a recent study of frailty (Gao et al., 2017, Epigenetics, 12 (2): 149-56), methylation at cg04987734 was associated with increased likelihood of mortality. This is remarkable because others have shown that demethylation at this locus is associated with smoking, we have also shown that cg04987734 as demethylated in those who drank heavily but did not smoke which suggests that the prior association of this locus with smoking may be due to the comorbidity of smoking and HAC.

A second reason that our findings have not been observed previously may be that the use of statins may obfuscate the ability of one of the most commonly used alcohol biomarkers, carbohydrate deficient transferrin (CDT), to detect HAC. In 2014, Vidali reported that statin use was associated with a false negative for CDT. Furthermore, it is well established that statins increase the global level of sialyation in a number of proteins. Therefore, because statins are ubiquitously prescribed for those with CHD, it is quite possible that prior investigations using the CDT failed to detect the presence of HAC for methodological reasons.

Finally, it is important to realize that alcoholism is a silent killer whose presence can be notoriously hard to detect in standard clinical interviews. At our academic medical center, the vast majority of patients are interviewed by medical students, residents and staff on separate occasions. Therefore, it is very likely that almost all of these patients and/or their relatives were asked repeatedly about their alcohol consumption. We have only the highest regards for the skill of our fellow UIHC clinicians and we like to note that the consent for many of these subjects was obtained through their legal authorized representative because the subjects were unable to tolerate or fully comprehend the formal consenting process. Given these and other constraints in the practice of clinical medicine, it may not be possible to use clinical histories to detect the presence of AUD in these individuals in similar situations. Instead, DNA methylation or perhaps other biomarker methods, such as the use of phosphatidylethanol (PEth) may be necessary to help establish the presence of an AUD.

Whatever the methods used, it is imperative that steps to detect and treat HAC in those presenting with ACS be taken. Current treatment guidelines mandate that other better-established drivers of CHD, such as smoking and hypercholesteremia, which are clinically modifiable, be addressed. Similarly, alcohol use disorders are treatable. But to do so most effectively, they must be detected. The current studies, which use clinically implementable MSdPCR techniques, suggest that in the future, the use of DNA methylation technologies may be one mechanism to achieve detection.

In summary, we report that over two thirds of subjects admitted for an episode of ACS in the context of CHD have epigenetic evidence of HAC.

Therefore, based on the results described herein, the methylation status of CpG site cg07375256 can be used to effectively determining whether or not an individual will suffer from, or is suffering from, severe alcohol withdrawal syndrome or complications therefrom. It is well known that one of the complications from severe alcohol withdrawal syndrome is seizures.

As described herein, an increase in methylation at cg07375256 is indicative of an increased likelihood that the individual will suffer from severe alcohol withdrawal syndrome or complications therefrom, whereas a decrease in methylation at cg07375256 is indicative of a reduced likelihood that the individual will suffer from severe alcohol withdrawal syndrome or complications therefrom. Typically, evaluating the methylation status (e.g., an increase or decrease in methylation) relies on the ratio of methylation, in this case, at the cg07375256 loci.

In some instances, the patient also can be assigned a number based on the Prediction of Alcohol Withdrawal Syndrome Scale (PAWSS) (Maldonado et al., 2015, Alcohol Alcohol, 50 (5): 509-18). The PAWSS is a 10-item scale used to triage patients for risk of alcohol withdrawal, with a score of 4 or greater predicting severe alcohol withdrawal.

In addition, the increase or decrease in methylation at cg07375256 can be used to determine whether the individual is treated for severe alcohol withdrawal syndrome or complications therefrom or is not treated for severe alcohol withdrawal syndrome or complications therefrom.

Also based on the results described herein, the methylation status of multiple CpG sites can be used to effectively determine whether an individual presenting with myocardial infarction (MI) or acute coronary syndrome (ACS) is suffering from heavy alcohol consumption (HAC) or alcohol use disorder (AUD). This method utilizes an alcohol T score (ATS), which is the average of the Zscores for cg02583484, cg04987734, cg09935388 and cg04583842 loci and the ratio of methylation at the cg07375256 loci. It would be understood that a Zscore is the value observed minus the average value of the controls divided by the standard deviation of the controls, which is effectively a measure of how many standard deviations a given observation is from the norm. In some instances, the ATS and the ratio of methylation at the cg07375256 loci can be used individually or in combination to determine whether or not the individual receives treatment for HAC, AUD, MI, ACS, or combinations thereof.

Methods of determining whether or not a particular CpG dinucleotide is methylated are known in the art. For example, DNA from the individual can be bisulfite converted, which converts unmethylated cytosines into uracils through deamination, while leaving methylated cytosines unchanged. The bisulfite-converted DNA then can be amplified, if desired, and contacted with at least one oligonucleotide that is complementary to the loci containing the CpG dinucleotide. In some instances, an oligonucleotide can be used that detects the CpG site in the methylated state; in other instances, an oligonucleotide can be used that detects the CpG site in the unmethylated state. Alternatively, the methylation status of a CpG dinucleotide at a particular loci can be determined using methylation sensitive digital PCR (MSdPCR).

Typically, the DNA that is examined for methylation is obtained from a biological sample from the individual. Representative biological samples include, without limitation, peripheral blood, lymphocytes, urine, saliva, and buccal cells.

The methods described herein also can be implemented on a computer. For example, measured data associated with the methylation status of CpG site cg07375256 in an individual can be obtained; a predictive score can be generated based on the measured data; and a likelihood of severe alcohol withdrawal syndrome or complications therefrom (e.g., seizures) in the individual can be providing based on the predictive score. Similarly, for example, measured data associated with the ATS and ZSCAN25 values in an individual can be obtained; a predictive score can be generated based on the measured data; and a likelihood that an individual presenting with myocardial infarction (MI) or acute coronary syndrome (ACS) is suffering from heavy alcohol consumption (HAC) or alcohol use disorder (AUD) can be providing based on the predictive score. The computer-implemented methods described herein can also include outputting a predicted level of severity of the alcohol withdrawal syndrome, heavy alcohol consumption or alcohol use disorder based on the predictive score.

In addition to the methods described herein, articles of manufacture are provided that can be used to practice the methods. For example, an article of manufacture can include at least one oligonucleotide for detecting the methylation status of cg0737525, or an article of manufacture can include the oligonucleotides necessary for determining the ATS (i.e., oligonucleotides for detecting the methylation status of cg02583484, cg04987734, cg09935388 and cg04583842) and ZSCAN25 (i.e., oligonucleotide for detecting the methylation status of cg07375256).

Articles of manufacture as described herein also can include one or more additional oligonucleotides for determining the methylation status of one or more of the loci described herein; reagents for bisulfite-converting nucleic acids; reagents for performing methylation sensitive digital PCR (MSdPCR); and/or one or more therapeutics for treating such individuals (e.g., phenobarbital; anticonvulsant treatment).

Articles of manufacture provided herein also can contain a package insert or package label having instructions thereon for using the oligonucleotides and any additional components to determine the methylation status of one or more loci in a sample.

In accordance with the present invention, there may be employed molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### Examples

### Example 1—Methods and Materials

The protocols for collecting HAC subjects whose entry and exit DNA methylation data are featured in this study has been previously described (Philibert et al., 2018, Genetics, 177 (5): 479-88). In brief, the subjects were recruited from one of three Iowa inpatient treatment facilities near Iowa City. Each of these facilities offers detoxification services followed by a 21-28 day inpatient alcohol treatment program. Any individual who: 1) is over the age of 18, 2) is capable of giving consent in English, 3) who was admitted to one of these facilities for treatment of current sustained alcohol dependence in the context of current alcohol intoxication and 4) expressed interest in our protocol to a member of their healthcare team was eligible for the study. Participants were not approached for intake into the study if they were still intoxicated or judged to be under the influence of other substances. After written informed consent for the study was obtained, each participant was interviewed with a series of instruments including the Substance Use Questionnaire. After the interview process was complete, each participant was then given a contact card and urged to contact the facility staff or the research assistant if they were interested in completing the exit interview that was held as close to the discharge date as possible. Fifty four of the 143 individuals who participated in the intake interview also completed the discharge interview which consisted of an updated Substance Use Questionnaire and phlebotomy.

Methylation data for the identification of quickly reverting loci was conducting using intake (T1) and discharge (T2) samples from 47 individuals with HAC admitted for the inpatient treatment of AUD. After preparation, genome wide methylation assessments using the Infinium MethylationEpic Version 1.0 were conducted by the Mayo Clinic Genomic lab (Rochester MN). The resulting data were then processed and cleaned using our normal procedures with a total of 824,807 probes for 45 pairs of DNA samples surviving quality control. The resulting M-values for these samples were then converted to beta values, and exported for further analysis.

The Alcohol Withdrawal Syndrome (AWS) subjects were patients who were admitted to the University of Iowa Hospital for the management of alcohol withdrawal. In brief, after detoxifying, subjects who were interested participating were approached by staff members and educated on the protocol, and if still interested, enrolled in the study. After informed consent was received, subjects were interviewed with a REDCap administered battery including the PAWSS, a modified version of the Substance Use Questionairre and a battery of AUD related modules from the PhenX project, then phlebotomized. Key outcome variables including the occurrence of seizures, amount of benzodiazepines (BZD) consumed were abstracted from the electronic medical record by an individual (BP) blind to biological measure outcomes. Conversion of BZD dose to diazepam equivalents was done according to the method of Salzman and colleagues.

Control subjects were recruited from the University of Iowa community using emails targeting abstinent or non-abstinent individuals. After informed consent was received, these subjects were also interviewed with a REDCap administered battery including the PAWSS, a modified version of the Substance Use Questionnaire and a battery of AUD related modules from the PhenX project, then phlebotomized.

ATS levels and MSdPCR assessment of cg07375256 methylation were conducted using reagents and software from Behavioral Diagnostics (Coralville, IA) and from Bio-Rad (Hercules, CA) using our previous methods (see, e.g., Philibert et al., 2019, J. Ins. Med., 48 (1): 90-102; Miller et al., 2020, Epigenetics, 16 (9): 969-79; Philibert et al., 2021, Genes, 13 (1): 2; Beach et al., 2022, Genes, 13 (10): 1888).

Data was analyzed using standard General Linear Models methods. Except where indicated, all p-values reported are nominal.

### Example 2—Experimental Results

Table 1 gives the clinical characteristic of the 45 subjects whose entry (T1) and exit (T2) DNA methylation values were used to identify fast responding DNA methylation loci. In brief, the subjects tended to be in their early 40's with approximately 90% of the sample being White (40 of 45). As per our 2019 publication, these subjects had all reported drinking almost every day for the eight weeks prior admission and ingested approximately 15 standard drinks per day in the month prior to admission.

TABLE 1
Clinical Characteristics of the 45 Subjects Whose
Data Were Used to Identify Candidate Markers

| | Male | Female |
|---|---|---|
| N | 35 | 10 |
| Age | 42.5 ± 11.8 | 42.1 ± 10.1 |
| Ethnicity | | |
| White | 31 | 9 |
| African American | 4 | 1 |
| Drinks per day over the past month | 14.4 ± 9.5 | 15.6 ± 13.4 |
| Days between DNA samples | 21.8 ± 4.7 | 22.3 ± 4.7 |

The methylation values from the 45 paired DNA samples were evaluated with respect to two criteria. The first criterion that the absolute difference in Beta values between the T1 and T2 time points be at least 5%. This is because MSdPCR values have a fixed lower limit of precision and we wished to maximize signal to noise ratios. The second criterion is that the changes in DNA methylation statistically significant. However, after Bonferroni correction, methylation at 18 loci were significantly different between intake and exit time points. Table 2 lists the 7 loci that had an absolute difference of DNA methylation values of 4.7% and were at least nominally different between study entry and exit.

TABLE 2
Identification of Candidate Loci for Abstinence-
Induced Reversion of Methylation

| | | | | | Previous |
|---|---|---|---|---|---|
| | | | | | Diff |
| Illumina ID | Gene | Difference | P-Value | Bonferroni | (2014) |
| cg27546431 | MYO1C | 0.074213 | 1.14E−05 | NS | 0.010645 |
| cg09807356 | GALNS | 0.066176 | 2.09E−10 | 0.000178 | 0.028749 |
| cg12389043 | CCDC92 | 0.052147 | 3.31E−07 | NS | 0.013004 |
| cg07692435 | | 0.051218 | 4.34E−06 | NS | |
| cg07375256 | ZNF498 | 0.051036 | 3.28E−07 | NS | 0.056552 |
| cg18424841 | | 0.049764 | 0.000133 | NS | 0.034717 |
| cg21281638 | C7orf26 | 0.046891 | 1.26E−05 | NS | |

We examined the differences in DNA methylation between the entry and exit found in those samples listed in Table 2. Interestingly, the directionality of change in methylation was the same at all five loci, with cg07375256 having an absolute difference of 5.6% between study intake and exit, suggesting that methylation at this locus reliably changed as a function of abstinence from alcohol.

### Example 3—Alc12 Methylation Related to Alcohol Withdrawal Syndrome and Seizures

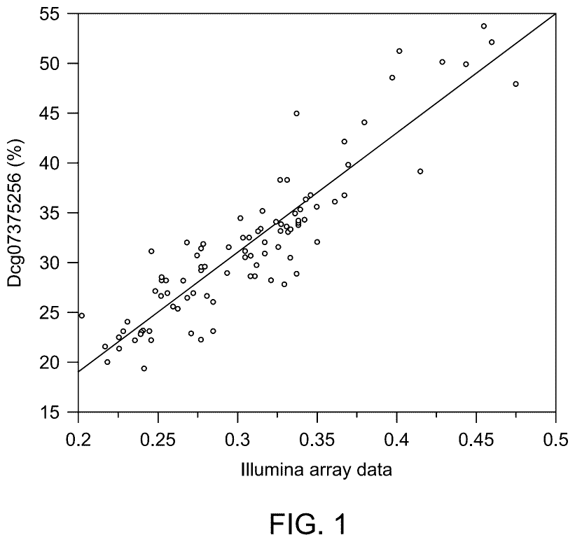

Using proprietary methods, we then designed fluorescent MSdPCR primer probe sets for cg07375256. FIG. 1 illustrates the relationship between DNA methylation per that MSdPCR assay (termed Dcg07375256) with the Illumina probe values at that locus in 90 samples of DNA from a previous study (2019). Overall, the correlation between the methylation values derived from the array with those from Dcg07375256 assay correlated well with a (r=0.92). Consistent with the development of prior MSdPCR markers, the range for the Dcg07375256 values was greater than that for the Illumina array.

Table 3 lists key demographic and clinical variables for the subjects in the study. Nearly 75% of the subjects were male (93 of 125) with both male and female subjects being in their mid-forties. The vast majority (94%) were White. The self-reported rate of lifetime smoking was high with nearly 60% of subjects reporting smoking at least 100 cigarettes in their lifetime. Although a noticeable fraction (18%) of the subjects report less than 5 drinks per day over the past month, nearly two-thirds (66%) reported drinking 10 drinks or more over the past month.

TABLE 3
Demographics

| | Cases | | | | Controls | | | |
|---|---|---|---|---|---|---|---|---|
| | Female | | Male | | Female | | Male | |
| Variable | (n = 32) | | (n = 93) | | (n = 31) | | (n = 50) | |
| Age in years (SD) | 47.4 | (15.4) | 44.4 | (13.3) | 42.3 | (12.5) | 32.3 | (13.7) |
| Hispanic | 1 | (3) | 5 | (5) | 3 | (10) | 4 | (8) |
| Race (may be > one) | | | | | | | | |
| Native American | 1 | (3) | 5 | (5) | 0 | (0) | 0 | (0) |
| Asian | 0 | (0) | 0 | (0) | 3 | (10) | 3 | (6) |
| African American | 1 | (3) | 3 | (3) | 1 | (3) | 1 | (2) |
| Native Hawaiian or | 0 | (0) | 0 | (0) | 0 | (0) | 0 | (0) |
| Pacific Islander | | | | | | | | |
| European American | 31 | (97) | 87 | (94) | 28 | (90) | 45 | (90) |
| Other | 0 | (0) | 2 | (2) | 0 | (0) | 1 | (2) |
| Prefer not to | 0 | (0) | 1 | (1) | 0 | (0) | 1 | (2) |
| answer | | | | | | | | |
| Ever smoked 100 | 13 | (41) | 60 | (65) | — | | — | |
| cigarettes or more | | | | | | | | |
| (%) | | | | | | | | |
| Smoked 1 PPD or | 4 | (13) | 22 | (24) | — | | — | |
| more in last month | | | | | | | | |
| (%) | | | | | | | | |
| Drinks per day in | | | | | | | | |
| past month (%) | | | | | | | | |
| 0 to 4 | 10 | (32) | 12 | (13) | 29 | (94) | 47 | (94) |
| 5 to 9 | 8 | (25) | 12 | (13) | 1 | (3) | 0 | (0) |
| 10 to 14 | 5 | (16) | 25 | (27) | 0 | (0) | 0 | (0) |
| 15 to 19 | 2 | (6) | 18 | (19) | 0 | (0) | 0 | (0) |
| 20 or more | 6 | (19) | 26 | (28) | 1 | (3) | 3 | (6) |
| Not reported | 1 | (3) | 0 | (0) | 0 | (0) | 0 | (0) |

| | | | | | | |
|---|---|---|---|---|---|---|
| Median days since | 2 | (1, 3) | 2 | (1, 3) | — | — |
| last drink (25, | | | | | | |
| 75% ile) | | | | | | |

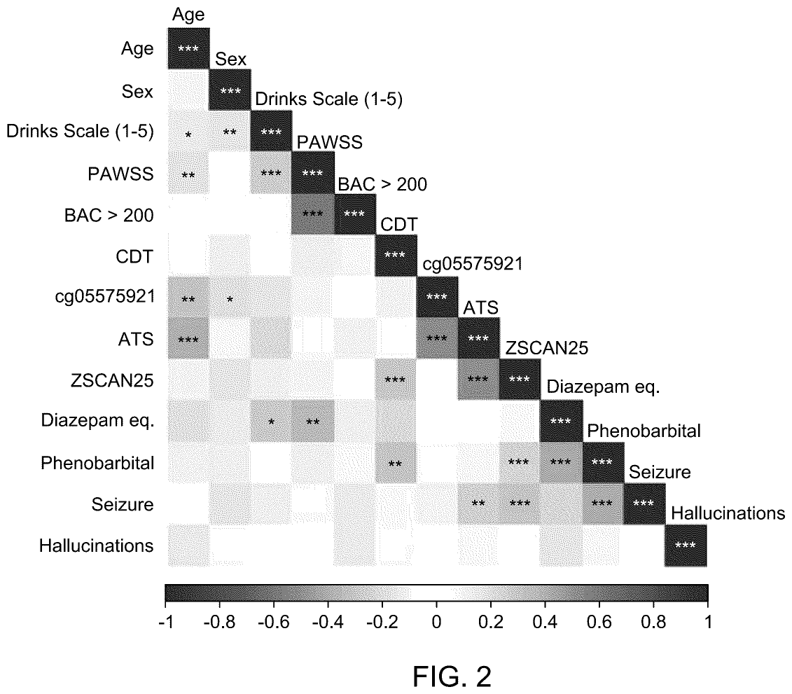

FIG. 2 illustrates the relationships between key study variables listed in Table 3. Age was significantly associated with drinks per day, PAWSS score, cg05575921 levels and ATS values. Biological sex was associated both with self-reported drinking and cg05575921 indicated smoking intensity. Self-reported drinking was associated with age, sex and PAWSS score. PAWSS levels were associated with age, self-reported drinking levels, having a BAC of greater than 200 mg/dl at admission and total benzodiazepine dosage. BAC levels were only associated with PAWSS scores. CDT levels were associated with Dcg07375256 (ZSCAN25) and phenobarbital administration. Deg05575921 levels were associated with ATS scores, CDT levels, phenobarbital administration, seizures. Diazepam dosing was associated with PAWSS and self-reported drinking levels. Phenobarbital administration was associated with CDT levels, Dcg05575921 values, and seizures. Seizures were associated with ATS scores, Dcg05575921 methylation and phenobarbital administration. Finally, the occurrence of hallucinations was not associated with any study variable.

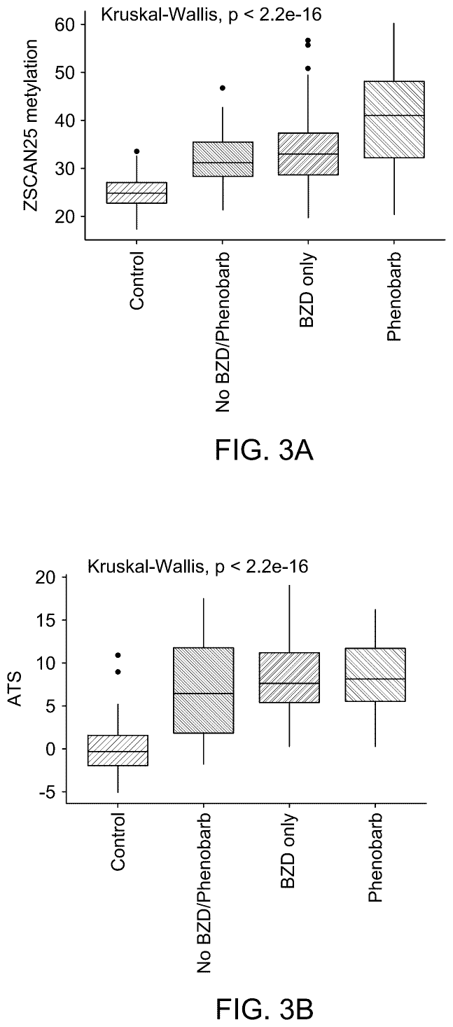

Our study attempted to use one of three predictor variables, PAWSS, ATS and Dcg07375256, to predict key clinical outcomes. The first outcome variable is the use of medications. FIG. 3A-3C illustrates the relationship between each of these three predictors and one of four treatment groups, 1) control, 2) observation without the use of medications, 3) use of just benzodiazepines or 4) use of phenobarbital with or without the use of benzodiazepines. PAWSS was associated with the use of benzodiazepines but not the use of phenobarbital. ATS levels were not associated with either treatment group. Finally, Deg07375256 levels were strongly associated with the use of phenobarbital.

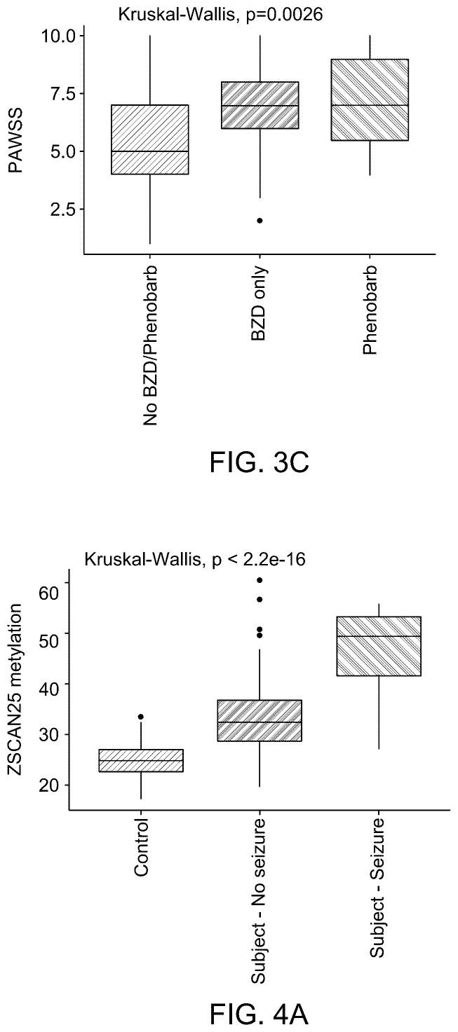

Nine alcohol related seizures were observed during the study. FIG. 4A-4C illustrates the relationship of PAWSS, ATS and Deg05575921 values to seizures. ATS was significantly and PAWSS, ATS and Dcg07375256 were highly significantly associated with the occurrence of seizures. In contrast, there was no relationship of PAWSS values to the occurrence of seizures.

### Example 4—ZSCAN25 and Alcohol T Score (ATS) Values are Markedly Elevated in Individuals Admitted for Acute Coronary Syndrome (ACS)

Within our data, we noted a strong relationship of alcohol T score (ATS) and carbohydrate deficient transferrin (CDT) values to the development of the epigenetic signature of coronary heart disease (CHD) in the Family and Community Health Studies (FACHS) population. This led us to hypothesize that the “stress” associated with heavy alcohol consumption (HAC)/alcohol use disorder (AUD) is a driver of myocardial infarctions (MIs). To test that hypothesis, we examined ATS and ZSCAN25 levels in DNA from our biobank of subjects admitted for acute coronary syndrome (ACS), almost all of whom have ST-elevated myocardial infarction (STEMI) or non-ST elevated myocardial infarction (NSTEMI).

What we found was nothing short of astounding. Nearly 20% (23 of 118) of the ACS subjects had ZSCAN25 levels predictive of severe alcohol withdrawal syndrome (AWS), while 64% had ATS levels of >3.5, which is predictive of heavy alcohol consumption (HAC). However, only 17 of the subjects had a diagnosis of AUD and only 3 were referred for alcohol treatment. We are obtaining CDT levels for the subjects with high ZSCAN25 levels.

In summary, these data provide compelling evidence that HAC is a major factor driving ACS presentations, raise concerns that many subjects admitted for ACS may be experiencing AWS and that their AUDs are not being clinically addressed. If so, these findings have considerable clinical and policy implications.

### Example 5—Methylation Studies to Correlate Heavy Alcohol Consumption with Acute Coronary Syndrome

Heavy alcohol consumption (HAC) is a well-established risk factor for the development of coronary heart disease (CHD). However, the relationship of HAC to acute coronary syndrome (HAC) in those with CHD is not well understood. To better understand this relationship, we examined DNA methylation biomarkers for both chronic HAC and Alcohol Withdrawal Syndrome in 172 subjects admitted to a large tertiary care hospital for the care of ACS associated with CHD. Remarkably, we found that 64% of the ACS subjects had Alcohol T Scores (ATS) indicative of current HAC while 15% had elevated levels of ZSCAN25 methylation, a marker of short-term alcohol consumption. Both ATS (p<0.04) and ZSCAN25 (p<0.05) levels were significantly in those who died with survival analysis showing a significant effect of both age at time of admission and ATS levels on survival. Remarkably, however, review of discharge diagnoses revealed that only 12% of the subjects had a current or past diagnosis of alcohol use disorders. These finding indicate that HAC is a marked driver of ACS presentations and that by addressing HAC prospectively, we may be able to reduce the morbidity and mortality of CHD.

Therefore, the values for our long-term (ATS) and short-term (for ZSCAN25) methylation alcohol consumption biomarkers were evaluated in 172 subjects admitted for ACS in the context of CHD.

### Methods

The clinical and biological data for these studies were obtained from the Iowa CHD Repository Study. All protocols and procedures used in this repository have been approved by the University of Iowa Institutional Review Board (IRB #201910834).

Each of these subjects in the current study were individuals over the age of 18 years who presented to the University of Iowa Hospital and Clinics (UIHC) and were hospitalized for the evaluation and treatment of ACS in the context of CHD. After informed consent was obtained from the subject or their legally authorized represented, each subject was interviewed, when possible, to confirm the CHD presentation history, and then underwent phlebotomy. After discharge, the charts were abstracted by research assistants for blinding to methylation status to confirm the clinical diagnosis of CHD and obtain the other data for this study.

Age matched control subjects without symptomatic CHD and self-reported alcohol use disorder were recruited by advertisements within the University of Iowa community. After informed consent was obtained from each of these individuals, they were interviewed with a variety of instruments including those from the PhenX toolkit and then underwent phlebotomy. When possible, subjects' report of an absence of CHD was confirmed using hospital records.

All subjects were phlebotomized to obtain whole blood that was processed into DNA using our standard procedures. DNA methylation assessments of cigarette and alcohol consumption were conducted using MSdPCR per our previously described procedures (REF 3 and 16). In brief, a quantitative assessment of smoking of cg05575921, a generally accepted biomarker of smoking intensity, was conducted using a MSdPCR assay (Behavioral Diagnostics, Coralville, IA) and droplet digital PCR machinery and reagents (Bio-Rad, Carlsbad, CA) according to manufacturer's instructions. Similarly, ATS values for each subject were determined using the Alcohol Signature assay (Behavioral Diagnostics, Coralville, IA) and droplet digital PCR machinery and reagents (Bio-Rad, Carlsbad, CA) as previously described. The Alcohol Signature is a battery of four assays that assess methylation status at loci, cg02583484, cg04987734, cg09935388 and cg04583842, which are highly responsive to alcohol consumption and are not affected by smoking. The ATS metric is then produced from these four assessments by calculating an unweighted sum of alcohol induced changes in the T-score (TS) at the four sites. In brief, the TS for each CpG site is produced by subtracting the previously established mean of methylation at the locus in abstinent controls from the observed methylation value for each subject at each locus, then dividing the result by the standard deviation of the controls at that locus.

The data were analyzed using the suite of general linear model analytic algorithms embedded in JMP Version 17 (SAS Institute, Cary SC). Comparisons of normally and non-normally distributed continuous variables between groups were conducted using T-Tests and Wilcoxon rank sum tests, respectively. Weibull survival analysis was used to determine the relationship of key variables to survival.

### Results

Table 4 delineates the clinical and demographic characteristics of 172 subjects who participated in this study. Consistent with the demographics of the State of Iowa and the epidemiology of ACS, the majority of subjects in this study were White with both sexes having an average age of 64±12 years. Although CHD strikes both males and females relatively equally, ⅔rds of the subjects were male (116 of 172).

Chart diagnoses of AUD were not frequent in this population. A review of the medical records charts showed that only 17 of the first 141 subjects (12%) had a current or past diagnosis of an alcohol use disorder. Only 6 of the first 141 subjects reported drinking more than 2 drinks per day prior to admission.

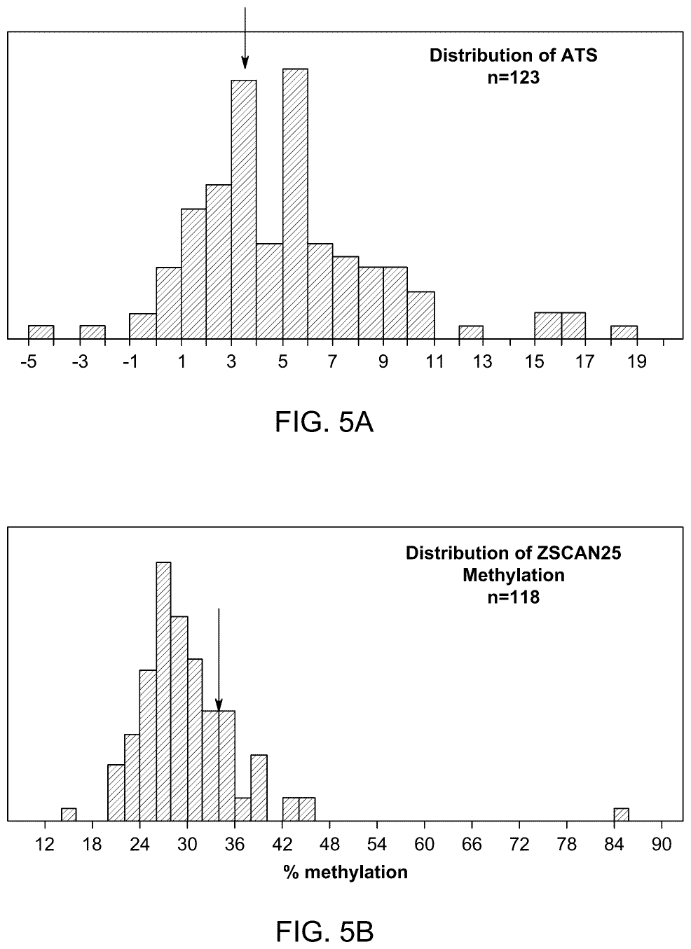

Table 4 and FIG. 6 delineate the distribution of ZSCAN25 and ATS values scores in these subjects. ZSCAN25 is a biomarker indicative of more recent consumption of alcohol. In our prior studies, the average methylation at this locus in a group of 47 self-reported abstinent was 28.2 was only 29.4% overall. Remarkably, there were 34 subjects (34 of 165, 21%) with ZSCAN25 levels of 34% or more which is suggestive of risk for severe alcohol withdrawal with one of the subjects having a remarkable level of 84%.

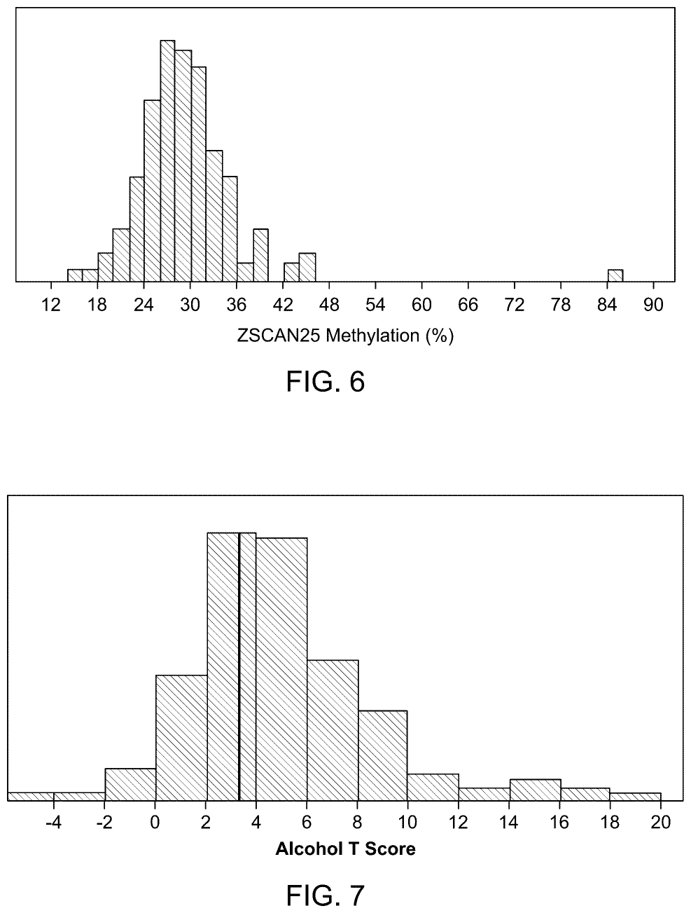

Table 4 and FIG. 7 delineate the distribution of the ATS in the same population. In abstinent populations, the ATS is a unitless, zero centered metric with a standard deviation of 2.2. However, in the 170 subjects for whom we have methylation assessments at this locus, ATS scores were globally elevated with average of 5.0±3.6. Remarkably, 80 of the 115 male subjects for whom we have measurements had ATS values of >3.5 which is strongly predictive of HAC.

TABLE 4
Clinical and Demographic Characteristics of Subjects

| | | Male | Female |
|---|---|---|---|
| N | | 116 | 56 |
| Age | | 64 ± 12 years | 64 ± 12 years |
| Death Status | | | |
| | Alive | 102 | 49 |
| | Dead | 14 | 7 |
| | Average Survival | 572 ± 523 days | 551 ± 551 days |
| | Observed | | |
| Epigenetic | | | |
| Assessments | | | |
| Smoking | | | |
| | cg05575921 < 80% | 63 | 16 |
| | cg05575921 ≥ 80% | 51 | 35 |
| | Missing | 2 | 3 |
| | cg05575921 | 66 ± 22% | 77 ± 17% |
| Drinking | | | |
| | ZSCAN25 | 30 ± 8% | 28 ± 5% |
| | Alcohol T Score | 5.2 ± 3.7 | 4.6 ± 3.4 |

In some studies, HAC consumption has been shown to be a risk factor for the development of CHD. However, whether its presence affects subsequent survival is unknown. To better understand this, we first contrasted the ZSCAN25 and ATS levels in those who died to those how lived. Values for ZSCAN25 (31.5±5.4% vs 29.1±6.9%, p<0.05) and ATS (6.5±3.3 vs 4.8±3.6, p<0.04) were significantly higher in those who died.

Next, we conducted standard parametric survival analysis of the cohort. As Table 5 demonstrates, a simple model of age and ATS values significantly predicted survival. Interestingly, the addition of sex, a traditional risk factor for CHD, or other parameters did not improve the model.

TABLE 5
Effect Likelihood Tests

| Source | Nparm | DF | ChiSquare | P value |
|---|---|---|---|---|
| Age | 1 | 1 | 7.5 | P < 0.0062 |
| ATS | 1 | 1 | 3.8 | P < 0.0500 |

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of determining whether or not an individual will suffer from severe alcohol withdrawal syndrome or complications therefrom, comprising the steps of:
determining the methylation status of CpG site cg07375256 in a biological sample from the individual;
wherein the methylation status of the at least one CpG dinucleotide identifies individuals that will suffer from severe alcohol withdrawal syndrome or complications therefrom.

2. The method of claim 1, wherein the complications from severe alcohol withdrawal syndrome comprise seizures.

3. The method of claim 1, wherein an increase in methylation at cg07375256 is indicative of an increased likelihood that the individual will suffer from severe alcohol withdrawal syndrome or complications therefrom.

4. The method of claim 1, wherein a decrease in methylation at cg07375256 is indicative of a reduced likelihood that the individual will suffer from severe alcohol withdrawal syndrome or complications therefrom.

5. The method of claim 1, wherein the determining step comprises methylation sensitive digital PCR (MSdPCR).

6. The method of claim 1, wherein the determining step comprises:
contacting DNA in the biological sample with bisulfite under alkaline conditions to produce bisulfite-treated DNA;
optionally, amplifying the bisulfite-treated DNA to produce amplified bisulfite-treated DNA;
contacting the bisulfite-treated DNA with at least one oligonucleotide that is complementary to a sequence comprising the at least one CpG dinucleotide; and
detecting the methylation status of the at least one CpG dinucleotide.

7. The method of claim 1, wherein the methylation status is determined using bisulfite treated DNA.

8. The method of claim 1, wherein the at least one oligonucleotide detects the CpG site in the methylated state.

9. The method of claim 1, wherein the at least one oligonucleotide detects the CpG site in the unmethylated state.

10. The method of claim 1, wherein the biological sample is selected from the group consisting of peripheral blood, lymphocytes, urine, saliva, and buccal cells.

11. The method of claim 1, further comprising assigning a number to the individual based on the Prediction of Alcohol Withdrawal Syndrome Scale (PAWSS).

12. The method of claim 1, further comprising treating the individual for severe alcohol withdrawal syndrome or complications therefrom.

13. The method of claim 1, further comprising not treating the individual for severe alcohol withdrawal syndrome or complications therefrom.

14. A method of determining whether an individual presenting with myocardial infarction (MI) or acute coronary syndrome (ACS) is suffering from heavy alcohol consumption (HAC) or alcohol use disorder (AUD), comprising the steps of:
determining the alcohol T score (ATS) (i.e., average of 4 Zscores for cg02583484, cg04987734, cg09935388 and cg04583842) for the individual; and
determining the methylation status of CpG site cg07375256 in a biological sample from the individual
wherein the ATS and the methylation status of CpG site cg07375256 are indicative of whether an individual presenting with MI or ACS is suffering from HAC or AUD.

15. The method of claim 14, wherein the determining step comprises methylation sensitive digital PCR (MSdPCR).

16. The method of claim 14, wherein the determining step comprises:
contacting DNA in the biological sample with bisulfite under alkaline conditions to produce bisulfite-treated DNA;
optionally, amplifying the bisulfite-treated DNA to produce amplified bisulfite-treated DNA;
contacting the bisulfite-treated DNA with at least one oligonucleotide that is complementary to a sequence comprising the at least one CpG dinucleotide; and
detecting the methylation status of the at least one CpG dinucleotide.

17. The method of claim 14, wherein the methylation status is determined using bisulfite treated DNA.

18. The method of claim 14, wherein the at least one oligonucleotide detects the CpG site in the methylated state.

19. The method of claim 14, wherein the at least one oligonucleotide detects the CpG site in the unmethylated state.

20. The method of claim 14, wherein the biological sample is selected from the group consisting of peripheral blood, lymphocytes, urine, saliva, and buccal cells.

21. The method of claim 14, further comprising treating the individual for HAC, AUD, MI and/or ACS.
